(19) United States
(12) Patent Application Publication
(10) Pub. No.: US 2024/0226355 A9
(43) Pub. Date: Jul. 11, 2024

(54) ACTIVE PLASMA STERILIZER WITH SMART CONTROL

(71) Applicant: SURFPLASMA, INC., Gainesville, FL (US)

(72) Inventors: Sarthak ROY, Gainesville, FL (US); Vladimir VICHNYAKOV, Gainesville, FL (US); Justin Thomas KOSKY, Gainesville, FL (US); Bhaswati CHOUDHURY, Gainesville, FL (US)

(21) Appl. No.: 18/489,374

(22) Filed: Oct. 18, 2023

Related U.S. Application Data
(60) Provisional application No. 63/417,521, filed on Oct. 19, 2022.
(60) Provisional application No. 63/417,672, filed on Oct. 19, 2022.

(51) Int. Cl. A61L 2/14

(52) U.S. Cl. CPC A61L 2/14; A61L 2202/122

(57) ABSTRACT

An active plasma sterilizer (APS) system for sterilization/decontamination is provided, including a sterilization box and a plurality of compact portable plasma reactors (CPPRs) disposed in the sterilization box and configured for generating surface dielectric barrier discharge (SDBD) for generating and distributing reactive oxygen and nitrogen species (RONS) such as ozone. Each of the plurality of CPPRs includes a reactor panel and a power supply circuit. The reactor panel includes one or more electrodes separated by a dielectric medium. The active plasma sterilizer (APS) system may include an ozone decomposition module having a heating element connected to a power supply and configured to decompose ozone by heating ozonated air; a power supply module for supplying power to the heating element; connecting pipes for providing paths for ozonated air to travel through; and an air pump configured to circulate the ozonated air generated.

### CROSS-REFERENCE TO RELATED APPLICATIONS

This application claims the benefit of U.S. Provisional Application Ser. No. 63/417,521, filed Oct. 19, 2022, and U.S. Provisional Application Ser. No. 63/417,672, filed Oct. 19, 2022, the disclosures of each of which are incorporated herein by reference in their entirety, including any figures, tables, and drawings.

### BACKGROUND

The imperative necessity of interplanetary contamination prevention in the context of space research missions is grounded in the dual objective of safeguarding the celestial body of interest against terrestrial biological contaminants, commonly referred to as “forward contamination,” and shielding Earth from potential extraterrestrial agents, denoted as “backward contamination.”

This critical mandate aligns with the advancing frontier of space exploration, demanding the development of technologies and practices dedicated to preserving the integrity of scientific investigations conducted in space. Planetary protection is the practice of preventing cross contamination between earth and any celestial body of interest in a mission, ensuring credibility of the data collected during space missions while protecting the earth from extra-terrestrial organisms (backward contamination) and vice versa (forward contamination).

Although contamination control technologies for planetary protection are established for uncrewed missions, scientists are exploring technologies for crewed missions where microbial mitigation have increased complexity levels due to risk of recontamination by human contact. Furthermore, existing technologies approved by National Aeronautics and Space Administration (NASA) and the European Cooperation for Space Standardization (ECSS) for uncrewed missions—dry heat microbial reduction (DHMR), Ethylene Oxide (EtO), and vapor phase hydrogen peroxide (VHP), have certain drawbacks. The EC SS reported that DHMR can damage heat sensitive materials while VHP can cause detrimental material alteration. The EtO systems involve long exposure times (2.5 to 12 hours) and carcinogenic residuals, making them restricted by regulatory bodies such as OSHA and EPA. Knowledge gaps related to required concentrations in VHP, it's delivery mechanism and material compatibility have also hindered its utilization in space missions. Thus, there is a need for alternative technologies that can overcome disadvantages of currently approved methods for uncrewed missions while addressing microbial mitigation in crewed missions.

Researchers have identified low temperature plasma (LTP) as a potential alternative to traditional decontamination methods used in planetary protection and other areas like food preservation and surface sterilization. Dielectric barrier discharge (DBD) is a type of LTP which has been proven effective against a variety of microbes including bacterial endospores which is crucial for planetary protection applications. DBD is the electrical discharge formed between two electrodes separated by a dielectric barrier when a high enough AC voltage is applied across them. As the name suggests, surface DBD (SDBD) is the DBD formed on a surface. SDBD is unique as it causes generation of reactive species while influencing the flow of the surrounding gas without employing any moving parts. DBD decontamination occurs through direct contact with discharge or indirect contact with reactive species formed by the discharge. Indirect DBD treatment is advantageous for treating hidden surfaces and surfaces relatively larger than the discharge area. When SDBD is generated in air, reactive oxygen and nitrogen species (RONS) are formed that can destroy micro-organisms like bacteria, viruses and fungi. One such species is ozone which is known to cause microbial inactivation and is the longest living species in comparison to other RONS formed during SDBD generation in air. The mechanism of such microbial inactivation occurs through a progressive set of complex reactions that lead to destruction of cellular surface, leakage of cellular surface, leakage of cellular components and cell lysis, finally inactivating the micro-organism.

The Committee on Space Research (COSPAR), in alignment with this pivotal objective, has undertaken the development and periodic revision of international policies for planetary protection, with a biennial update cycle. These policies are based on the celestial body of interest and the type of mission. Planetary protection necessitates decontamination of assembly cleanroom facilities, space research equipment and spacecraft components for reduction (disinfection) or eradication (sterilization) of microbial inhabitants.

Decontamination technologies currently approved for space missions by the National Aeronautics and Space Administration (NASA) and the European Cooperation for Space Standardization (ECSS) include dry heat microbial reduction (DHMR) and vapor phase hydrogen peroxide (VHP). The Viking spacecraft test era led to the evolution and validation of decontamination processes using a combination of DHMR and solvent cleaning.

However, these technologies can be detrimental to advanced materials and electronics used in spacecraft development today. The ECSS reported that high processing temperatures used in DHMR lead to the damage of heat sensitive materials while hydrogen peroxide used in VHP can lead to detrimental material alteration. Further, despite of VHP being a NASA approved technology, knowledge gaps related to required concentrations, delivery mechanism and material compatibility are setbacks that hinder its utilization in space missions.

Consequently, there exists an exigent need for the exploration and development of alternative decontamination technologies that can surmount the deficiencies inherent in currently approved methodologies in the context of space missions.

### BRIEF SUMMARY

There continues to be a need in the art for improved designs and techniques for contamination control system and method for space applications.

According to an embodiment of the subject invention, an active plasma sterilizer (APS) system for sterilization/decontamination is provided, comprising a sterilization box and a plurality of compact portable plasma reactors (CPPRs) disposed in the sterilization box and configured for generating surface dielectric barrier discharge (SDBD) for generating and distributing reactive oxygen and nitrogen species (RONS). The RONS includes ozone. The sterilization box is made of polycarbonate. The sterilization box comprises a grid structure disposed inside the sterilization box and configured for decontamination measurement. The sterilization box comprises a plurality of through-holes disposed on lateral sides of the sterilization box, allowing suspension of inoculated coupons inside the sterilization box. Each of the plurality of CPPRs comprises a reactor panel and a power supply circuit. The reactor panel comprises one or more electrodes separated by a dielectric medium. The power supply circuit is configured to convert a low DC voltage to a high AC voltage for generating SDBD on surfaces of the reactor panel. The plurality of CPPRs comprise two reactor panels configured to generate different flow actuation for better distribution of the generated SDBD in the sterilization box. The two reactor panels include a comb reactor panel and a fan reactor panel, wherein the comb reactor panel is configured to generate a two-dimensional (2D) flow distribution with a dominant wall jet in a direction from a shaft towards teeth tips of the comb reactor panel, and wherein the fan reactor panel is configured to generate a three-dimensional (3D) flow distribution forming a swirl flow spreading vertically upwards and outwards from a center of the fan reactor panel. The plurality of CPPRs comprise three CPPRS each comprising a fan reactor panel and the three a fan reactor panels are positioned in an equilateral triangle formation on a top internal surface of the sterilization box, and one CPPR comprising a comb reactor panel disposed at a bottom surface of the sterilization box with a predetermined distance offset from a center of the bottom surface. The sterilization box can be, for example, a 3D-printed box using any suitable material (e.g., polycarbonate or any other suitable 3D printing material).

According to certain embodiment, an ozone decomposition system is provided, comprising: a heating element connected to a power supply and configured to decompose ozone by heating ozonated air; a power supply module for supplying power to the heating element; connecting pipes for providing paths for ozonated air to travel through; and an air pump configured to circulate the ozonated air generated. The ozone decomposition system may further comprise one or more sensors configured to measure ozone level or an insulation element for insulating heat generated by the heating element. The power supply module is a standard wall supply module or a battery powered module. The power supply module comprises feedback loop to supply power to the heating coil. The power supply module further comprises a power amplifier and a controller configured to control an input voltage to the power amplifier. An input voltage of the power supply module is supplied in a duty cycle. The power supply module further comprises feedback circuitry configured to monitor an operational parameter of a load and provide feedback to the controller, wherein the controller is configured to control operation of the power amplifier based on the provided feedback. The feedback circuitry comprises an ozone sensor configured to detect ozone in airflow.

According to another embodiment, an active plasma sterilizer (APS) system for sterilization/decontamination is provided, comprising a sterilization box; a plurality of compact portable plasma reactors (CPPRs) disposed in the sterilization box and configured for generating surface dielectric barrier discharge (SDBD) for generating and distributing reactive oxygen and nitrogen species (RONS); and an ozone decomposition system having a heating element connected to a power supply and configured to decompose ozone by heating ozonated air, a power supply module for supplying power to the heating element, connecting pipes for providing paths for ozonated air to travel through, and an air pump configured to circulate the ozonated air generated.

### BRIEF DESCRIPTION OF DRAWINGS

FIG. 1 is a schematic representation of APS sterilization box showing decontamination measurement locations, according to an embodiment of the subject invention.

FIGS. 2A-2F are schematic representations of smoke flow visualizations demonstrating reactor flow distribution that facilitates distributing ozone generated, wherein FIG. 2A shows a Comb reactor in which solid and dashed lines representing the exposed and ground electrodes, respectively; wherein FIG. 2B shows Comb reactor's characteristic flow in which arrows show three wall jets with the dominant one in the direction from the shaft towards the teeth tips; wherein FIG. 2C shows smoke flow visualization of the Comb reactor's actuated flow; wherein FIG. 2D shows a Fan reactor in which colored and grey areas representing the exposed and ground electrodes, respectively; wherein FIG. 2E shows the Fan reactor's characteristic flow of each blade and the center; and wherein 2F shows smoke flow visualization of the Fan reactor's actuated flow where the fan blades interact to form vortical structures resulting in overall conical flow thrusted upwards from the reactor base, according to an embodiment of the subject invention.

FIG. 3 shows images of process of integration of CPPRs and their placement inside the sterilization box, according to an embodiment of the subject invention.

FIG. 4 is a schematic representation of the measurement grid for decontamination measurement in three planes, according to an embodiment of the subject invention.

FIG. 5 is a schematic representation showing dimensions of the enclosure built for ozone penetration tests, according to an embodiment of the subject invention.

FIG. 6 shows images of Orthofabric (left) and Kevlar (right) Fabric Cells, according to an embodiment of the subject invention.

FIG. 7 shows plot diagrams of results establishing consistent controls with E. coli inoculation, that is, inoculating coupons of same material with same volume of E. coli culture resulted in consistent bacterial count per coupon with a maximum variation of 0.3 log 10 (CFU/coupon), according to an embodiment of the subject invention.

FIG. 8 shows plot diagrams of results establishing consistent controls with B. subtilis inoculation, that is, inoculating coupons of same material with same volume of B. subtilis culture resulted in consistent bacterial count per coupon with a maximum variation of 0.3 log 10 (CFU/coupon), according to an embodiment of the subject invention.

FIG. 9 shows plot diagrams of results showing logs of E. coli CFUs/coupon of exposed coupons (averaged over 11 coupons placed inside the APS) and control coupons (averaged over 3 coupons placed outside the APS) for four coupon materials, each repeated three times. Complete killing (4 to 5 log reduction) was achieved in each case using 4 CPPRs and exposures of 20 minutes (CPPRs on for 15 minutes and CPPRs off for 5 minutes), according to an embodiment of the subject invention.

FIG. 10 shows plot diagrams of results showing uniform spatial distribution of reduction in logs of E. coli CFUs/coupon of 9 exposed coupons placed at the central plane of the APS, for each material type. Complete killing (4 to 5 log reduction) was achieved at all points using 4 CPPRs and exposures of 20 minutes (CPPRs on for 15 minutes and CPPR off for 5 minutes), according to an embodiment of the subject invention

FIG. 11 shows plot diagrams of results showing logs of B. subtilis CFUs/coupon of exposed coupons (averaged over 11 coupons placed inside the APS) and control coupons (averaged over 3 coupons placed outside the APS) for four coupon materials, each repeated three times. Complete killing (4 to 5 log reduction) was achieved in most cases using 6 CPPRs and exposures of 30 minutes (CPPRs on for 25 minutes and CPPRs off for 5 minutes), according to an embodiment of the subject invention

FIG. 12 shows plot diagrams of results showing uniform spatial distribution of reduction in logs of E. coli CFUs/coupon of 9 exposed coupons placed at the central plane of the APS, for each material type. Complete killing (4 to 5 log reduction) was achieved at all points using 4 CPPRs and exposures of 20 minutes (CPPRs on for 15 minutes and CPPRs off for 5 minutes), according to an embodiment of the subject invention

FIG. 13A shows a plot diagram demonstrating APS ozone data for 4 CPPRs and 20 minutes exposure (CPPRs on for 15 minutes and CPPRs off for 5 mins), according to an embodiment of the subject invention.

FIG. 13B shows a plot diagram demonstrating APS ozone data for 6 CPPRs and 30 minutes exposure (CPPRs on for 25 minute and CPPRs off for 5 minutes), according to an embodiment of the subject invention.

FIG. 14 shows plot diagrams of results demonstrating ozone concentrations before and after fabric layers showing ozone penetration without and with pump, according to an embodiment of the subject invention.

FIG. 15 shows material compatibility data by SEM analysis of APS exposure to aluminum, Polycarbonate, Orthofabric and Kevlar, according to an embodiment of the subject invention.

FIG. 16 is a schematic representation showing embodiment of the ozone decomposition module (ODM) for achieving testing results, according to an embodiment of the subject invention.

FIG. 17 shows results of ODM embodiment testing for 0 seconds of ODM operation.

This represents the natural decay rate of the ozone at room temperature, according to an embodiment of the subject invention.

FIG. 18 shows results of ODM embodiment testing for 30 seconds of ODM operation, according to an embodiment of the subject invention.

FIG. 19 shows results of ODM embodiment testing for 60 seconds of ODM operation, according to an embodiment of the subject invention.

FIG. 20 is a schematic representation showing details of ODM, according to an embodiment of the subject invention.

FIG. 21 is a schematic representation showing a high voltage power supply module, according to an embodiment of the subject invention.

### DETAILED DESCRIPTION

Embodiments of the subject invention pertain to an Active Plasma Sterilizer (APS) system for surface decontamination for space and terrestrial applications.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In one embodiment, an Active Plasma Sterilizer (APS) system and methods are developed based on non-thermal plasma for addressing the disadvantages related to currently approved methods including temperatures and material compatibility, while providing a compact, lightweight, safe, rapid and economical decontamination solution.

Moreover, an ozone decomposition module (ODM) is developed, comprising a heat coil, electronics, and piping to provide a path for ozonated air to travel through. The heating coil heats the ozonated air as it passes through. Heating the air increases the decomposition rate of the ozone in the air. The electronics supply power to the heating coil, allowing it to operate at with a reasonably low power. An air pump such as a fan is used to force or pull out the ozonated air from the ozone generation chamber through the ODM and recirculated as many times as needed. The flowrate of the air pump can be in a range between 10⁻³ L/min to 10⁶ L/min.

The Active Plasma Sterilizer (APS) system comprises a central module having a microcontroller and relays to automatically control the frequency and power state of submodules. Moreover, the APS system comprises a set of sensors to find and maintain the most efficient frequency for each submodule based on feedback voltage and current of the submodules as well as the temperature, humidity, and ozone concentration within the sterilization chamber.

In one embodiment, it is not required to have any inductor in the APS system. Further, the APS system allows for automated locking and unlocking of the chamber once the sterilization cycle starts until the chamber reaches OSHA-approved and NIOSH-approved limits for concentrations of gases such as ozone. The system can comprise a door lock for safety of operation of the APS system, and the door lock can be an electromagnetic and/or mechanical door lock. The central module has indicators for each submodule connected and routes the power to the submodules from the main power supply.

The APS system and methods of the subject invention are based on surface dielectric barrier discharge (SDBD) which is a type of non-thermal plasma for generating and distributing reactive oxygen and nitrogen species (RONS), mainly ozone, for decontamination.

SDBD is produced by miniature reactors such as Compact Portable Plasma Reactors (CPPRs) that are compact, lightweight and energy efficient. The CPPRs adopt the Fan and Comb SDBD configurations capable of generating contrasting flow actuation and can be strategically integrated into the APS system and methods to achieve uniform spatial sterilization.

Design and fabrication of the APS system and methods of the subject invention are provided. Results show that a particular embodiment of the APS can achieve 4 to 5 log reductions of pathogenic bacteria such as Escherichia coli and Bacillus subtilis on materials including, but not limited to, aluminum, Polycarbonate, Kevlar and Orthofabric, simultaneously at 11 points distributed inside the APS, with a maximum ozone concentration×time (CT) requirement of 10323 ppm-minute, within 30 minutes with a power consumption of 13.2±2.22 W. Spatial distribution of the sterilization data at the central plane of the APS establishes that it can uniformly sterilize several areas of a contaminated surface within 30 minutes. Successful ozone penetration through Kevlar and Orthofabric layers is established by the CPPR in an enclosure with a maximum reduction of 16.17% in ozone concentrations through the layers without using an external agent to assist penetration.

In addition, preliminary material compatibility tests with SEM analysis of the aforementioned materials, when subjected to ozone CT values necessary for sterilization in the APS, reveal no substantial material damage.

Therefore, the APS technology, when applied in planetary protection scenarios, presents several advantages, including uniform spatial decontamination, low processing temperatures, short exposure durations, a lightweight design without moving parts, the capacity to decontaminate porous surfaces, and compatibility with materials relevant to space missions.

According to the embodiments of the subject invention, the Active Plasma Sterilizer (APS) system comprises dry, versatile, reusable and modularly scalable Compact Portable Plasma Reactors (CPPRs) which produce SDBD using a few watts of electrical power across electrodes separated by a dielectric medium. The APS system is based on the concept of achieving spatially distributed decontamination using a synergistic combination of SDBD ozone generation and flow actuation for distributing and mixing generated ozone without using external mixing agents. As a result, utilization of ozone generated for rapid decontamination is maximized while ozone requirements and residual concentrations are reduced. Thus, the APS system is designed to achieve spatially distributed decontamination using SDBD reactors. An APS prototype is evaluated by determining its sterilization efficacy, ozone CT (concentration×time) requirements, power requirements, penetration capability through desired materials and material compatibility. The test results show that the APS prototype can achieve complete killing (4 to 5 log reductions) of pathogenic bacteria such as Escherichia coli and Bacillus subtilis) on 4 selected materials: aluminum, polycarbonate, Kevlar, and Orthofabric, simultaneously at 11 points inside the chamber, with a maximum ozone CT requirement of 10323 ppm-minute, within 30 minutes using 13.2 W total power consumption. Additionally, successful ozone penetration for single and combined fabric layers is established without using an external agent. Further, preliminary material compatibility tests with SEM analysis of 4 selected materials exposed to ozone CT values required for sterilization in the APS system show no significant material damage.

### Materials and Methods

### Design of the APS System:

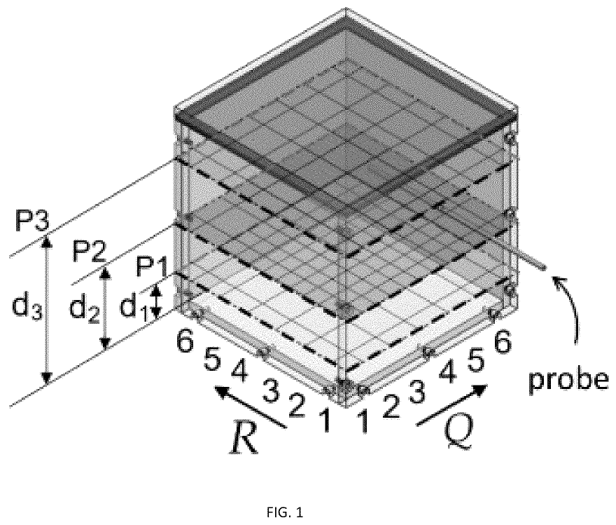

Sterilization Box: Referring to FIG. 1, a custom polycarbonate box with internal dimensions of, for example, 1 foot long, 1 foot wide, 1 foot high and wall thickness of 0.0065 foot is built. A layer of foam insulation adhesive is placed between the lid and sidewalls of the box to seal it from the external environment. Polycarbonate is chosen as it is non-reactive to ozone. For measuring decontamination achieved at internal points of the chamber, holes are drilled in the sidewalls to facilitate suspension of inoculated coupons inside the box as shown in FIG. 1. In one embodiment, three planes are selected to represent volume occupied by an object placed in the box: Plane 1 (P1), Plane 2 (P2) and Plane 3 (P3) positioned at d1=8 cm, d2=11.5 cm and d3=16 cm from the bottom of the box, respectively. The (P, Q, R) coordinate system is used to explain the decontamination measurement grid a later section. The drilled holes also provided access to a measurement probe for ozone data collection inside the box.

Compact Portable Plasma Reactor (CPPR): The CPPR incorporated into the APS sterilization box is designed to be compact and portable. Two components of the CPPR are: (a) the reactor panel comprising two sets of copper electrodes separated by a dielectric medium, and (b) the compact power supply circuit which converts a low DC voltage to a high AC voltage required for generating surface dielectric barrier discharge on the reactor panel surface. In a particular embodiment, the copper electrode sets are 35 μm thick separated by a dielectric material such as hydrocarbon/ceramic (RO4350B) composite having a thickness of 0.76 mm and a dielectric constant of 3.48. The CPPR power supply, also known as the active plasma module, is a compact circuit module with a volume of, for example, 48 cubic centimeters and a weight of 55 grams. The CPPR runs on 25V DC power supply and averagely consumes power of 2.2+0.37 Watts.

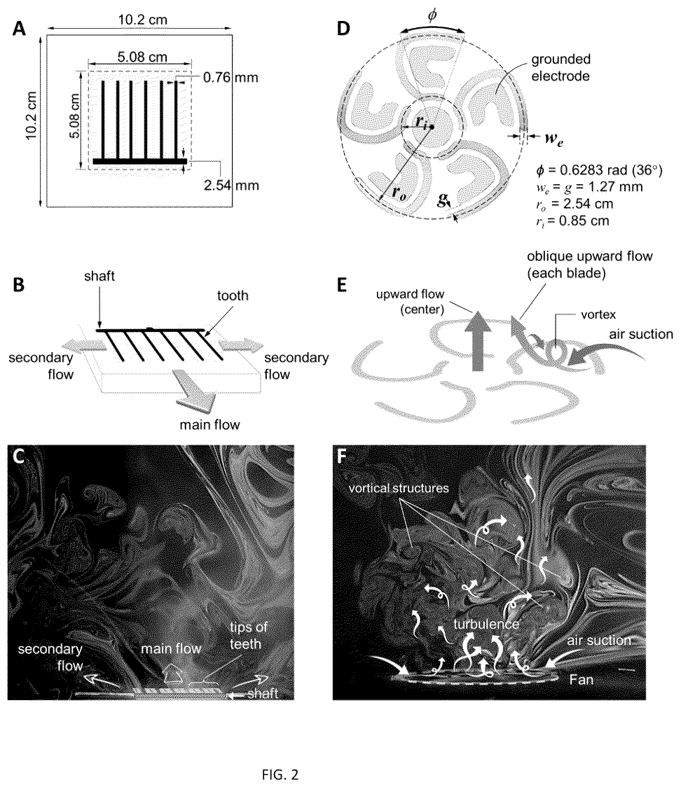

The power supplies are encased in electrically insulating cases for additional safety. The CPPR reactor panel is designed based on its flow actuation capabilities for distributing the ozone generated. Two reactor panel designs with contrasting flow actuation capabilities are used for better distribution of the generated ozone in the sterilization box: the comb reactor panel and the fan reactor panel. The two panel designs are shown in FIGS. 2A-2F. The comb reactor panel generates a two-dimensional (2D) flow distribution with a dominant wall jet in the direction from the shaft towards the teeth tips, while the fan reactor generates a three-dimensional (3D) flow distribution forming a swirl flow spreading vertically upwards and outwards from the center of the panel. Geometric details of the reactor panels are same as those used for ozone studies in the development of the SDBD Fan configuration.

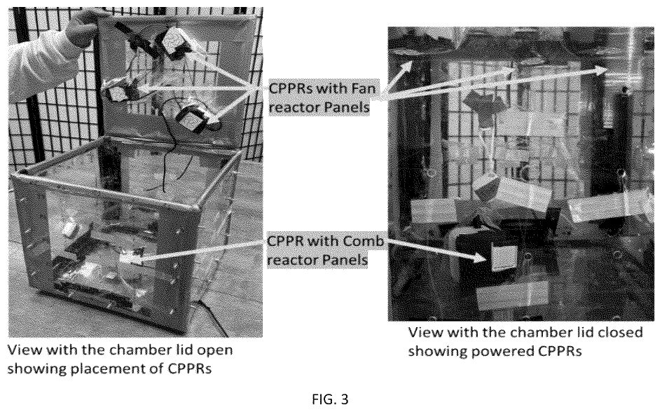

Integration of the CPPR into the Sterilization Box: A plurality of, for example, 4 CPPRs are integrated in the sterilization box as shown in FIG. 3. The placement and orientation of the reactors are based on the expected ozone distribution each CPPR reactor panel produces. Three CPPRs, each equipped with a fan reactor panel, are positioned in an equilateral triangle formation on the chamber's lid. This arrangement is designed to create vortical showers of ozone within the chamber's internal volume. Moreover, an additional CPPR with the comb reactor panel is placed at the bottom of the chamber with a 2 cm offset from the center. The comb reactor panel is configured to be placed such that the dominant wall jet from the shaft towards the teeth tips is directed upwards, ensuring ozone transportation from the bottom to the top to diffuse through the internal volume of the chamber. The offset is designed for inhibiting blocking of the wall jet by the coupons placed at the center points in planes 1, 2 and 3. The integration of the CPPRs into the sterilization chamber is carried out such that the number of CPPRs powered up during the experiments can be controlled.

### High Voltage Power Supply Module

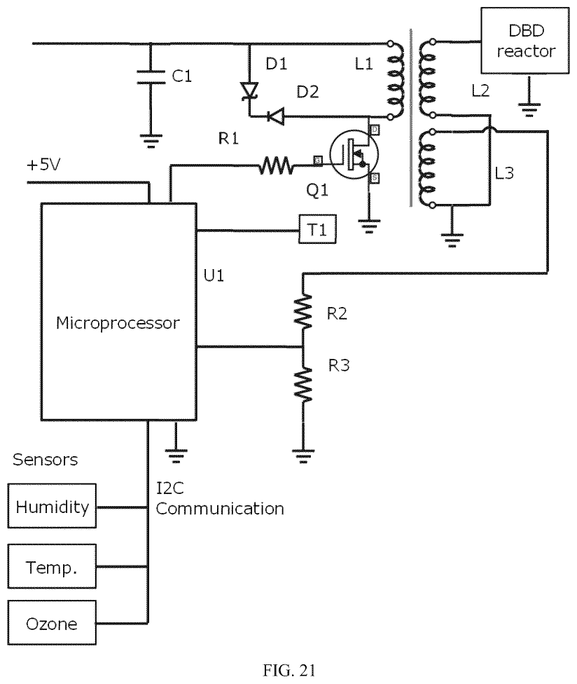

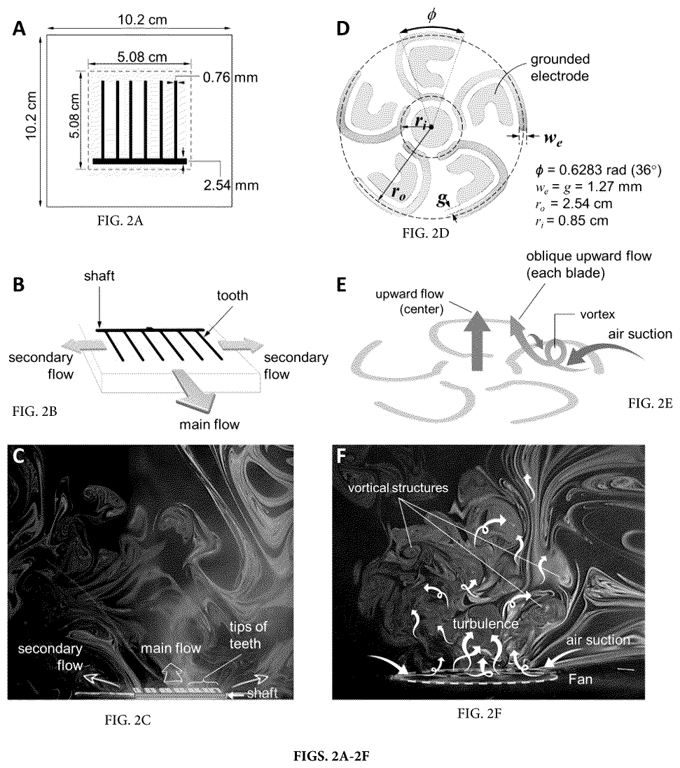

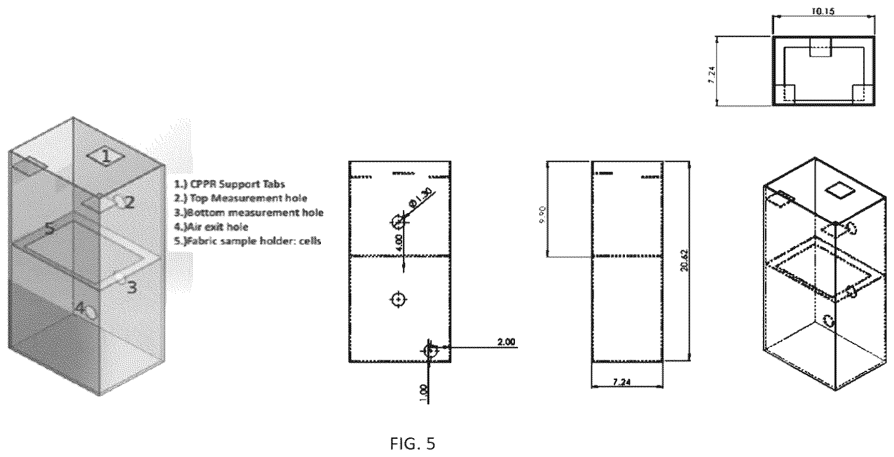

As shown in FIG. 21, a high voltage power supply module of the APS system is a programmable-microprocessor-based system. The control logic of the high voltage power supply module is configured for initial system tuning at power-up and continuous real-time monitoring based on advanced adaptive control method for optimal plasma generation for various loads.

During the initial setup of the system, the microprocessor is configured to determine the resonant frequency of the reactor and transformer without plasma generation. In this state, the gate pulse is selected to be sufficiently short to inhibit plasma ejection.

Then, the microprocessor is configured to scan the system from frequency f1 to f2. At the end of the scan, the microprocessor is configured to determine the maximum voltage on coil L3 and corresponding frequency. The frequency is next adopted to trigger the plasma generation. The gate pulse changes accordingly depending on the overall gate charge of the MOSFET.

During the ozone generation process, the program controls the L3 voltage and the rate of increase in ozone concentration. When the rate of ozone concentration begins to change due to changes in the load on the reactor, the microprocessor is configured to enter into an active tuning mode for changing the resonant frequency and gradually bring ozone production to the optimal level.

In one embodiment, the high voltage power supply module can provide power to at least one load. In another embodiment it can power multiple loads. A Wi-Fi chip may be connected through the I2C communication in the same way as the sensors.

In FIG. 21, C1 represents the current-sustaining capacitor of the MOSFET transistor (Q1); D1 and D2 represent the MOSFET transistor drain protection; L1, L2, and L3 represent primary, secondary and tertiary windings of the transformer (Tr1), respectively; Q1 represents a power MOSFET transistor; R1 represents a gate resistor; R2 and R3 represent voltage divider; T1 represents MOSFET temperature sensor; U1 represents microprocessor; +24 V represents built-in short circuit protection; +5V represents microprocessor power supply; and sensors include sensors configured for measuring humidity, temperature, and ozone level, respectively.

### Evaluation of Efficacy of the APS in Sterilizing Contaminated Materials of Interest

Experiments are performed to test sterilization efficacy of the APS system, including tests performed for (a) 11 measurement points in the sterilization box, (b) two test organisms commonly used for sterilization tests, (c) four materials commonly used in spacecraft applications, (d) up to 6 CPPRs to determine a minimum number of CPPRs required to obtain sterilization within 30 minutes, and (e) 7 exposure timepoints. Each of these tests is described below, followed by an explanation of the experimental procedure.

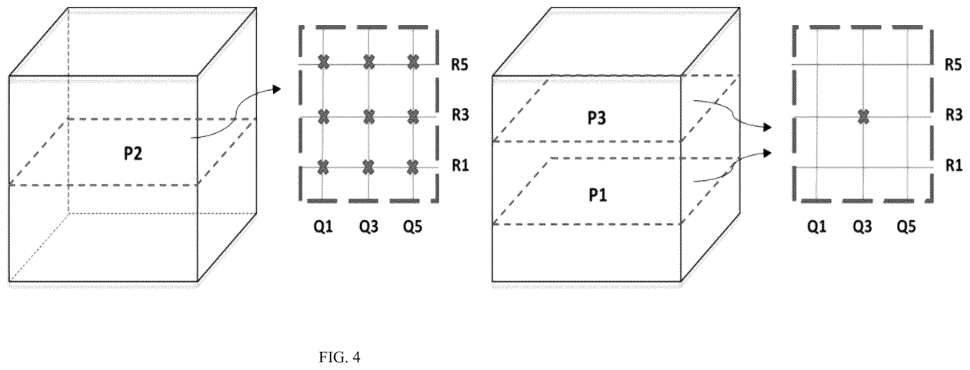

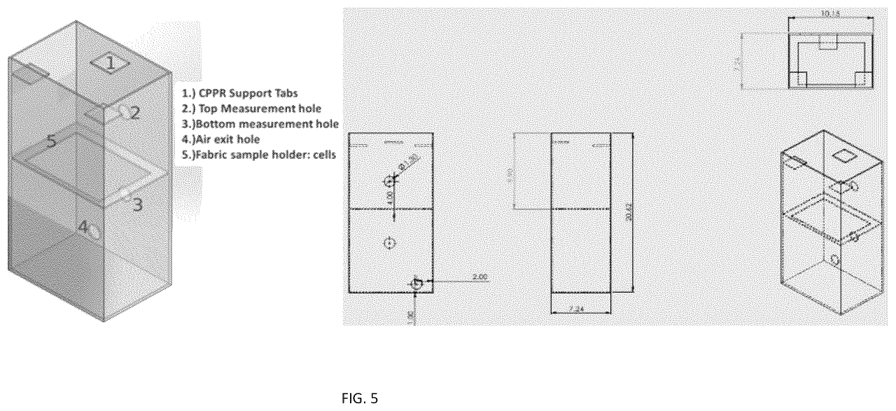

Measurement Grid: 11 measurement points are selected in the internal volume of the prototype to simulate distributed decontamination of various points on the surface of an object placed inside the box, including 9 points on Plane P2 (central plane) and centers of planes P1 and P3 as shown in FIG. 4. For suspending coupons inside the chamber in the measurement grid, sterile Teflon coated strings (for example, 0.1 mm diameter) are used to inhibit ozone loss, as Teflon does not react with ozone.

Test Organisms: Escherichia coli and Bacillus subtilis

Escherichia coli (ATCC 11775) NCTC 9001 Escherichia coli (Migula) Castellani and Chalmers, Serovar O1:K1:H7, Type strain. ATCC 11775 (BSL 2 level): Escherichia coli (E. coli), a gram-negative bacterium, is a rod-shaped, facultative anaerobe with a replication ability under unfavorable conditions, making the species suitable for the evaluation of decontamination technologies.

Bacillus subtilis (ATCC 6051) (Ehrenberg) Cohn, Type strain, Bacteriophage host (BSL 1 level): Bacillus subtilis (B. subtilis), is a spore forming gram positive aerobe generally found in soil and vegetation. Although non-pathogenic, it can contaminate food and be pathogenic for immuno-compromised people. The bacterial species is chosen as it is widely used in disinfection studies employing traditional disinfection methods. Moreover, due to its spore forming ability, the species is of interest in evaluating decontamination technologies for planetary protection.

Test Materials: Four materials are selected for testing due to their important roles in space missions. These materials are cut into 1-square-inch coupons, and they are presented in the table below along with referenced usage examples.

TABLE 1
Materials selected to be tested:

| Materials | Example Usage |
|---|---|
| aluminum 6060 | structural |
| Kevlar | thermal protection, EMU suits |
| Polycarbonate | EMU suits (visor) |
| 116 Ortho-Fabric | EMU suits |

Number of CPPRs and Exposure Times: Exposure times refer to the period for which the inoculated coupons are placed inside the APS prototype. Following combinations of number of CPPRs and exposure times (CPPR ON+CPPR OFF times) are iteratively tested to determine the number of CPPRs required to achieve complete killing of the selected bacteria species.
3 CPPRs: 10 mins (ON throughout).
4 CPPRs: 5 mins (ON throughout).
4 CPPRs: 15 minutes (10 mins ON+5 mins OFF)
4 CPPRs: 20 minutes (15 mins ON+5 mins OFF)
4 CPPRs: 25 minutes (20 mins ON+5 mins OFF)
4 CPPRs: 30 minutes (15 mins ON+5 mins OFF+5 mins ON+5 mins OFF)
6 CPPRs—30 minutes (25 mins ON+5 mins OFF)

Preparation of Cultures: Stocks of E. coli and B. subtilis strains are stored at −80° C. in LB (Luria-Bertani) broth and Nutrient broth, respectively, with 30% glycerol. Frozen stocks of E. coli and B. subtilis are grown overnight in LB broth at 37° C. and Nutrient broth at 30° C., respectively. A Spectrophotometer is used for estimating the concentration of bacteria in the fresh LB or nutrient broth culture, followed by dilution, if necessary, to obtain a concentration of approximately 5×10⁷ CFU (colony forming units)/ml. Next, 10 μl of these broth cultures is used to inoculate coupons of the selected materials with 4 to 5 logs of E. coli and B. subtilis.

Pre-processing of Coupons and APS box: The coupons are sterilized by autoclaving at 121° C. in a dry autoclave cycle. At the beginning of each experiment, the box and all the components inside it are wiped with 70% isopropyl alcohol to inhibit external contamination. Further, it is ensured that ozone concentrations inside the box prior to starting an experiment matched room level ozone concentrations by giving at-least half an hour of free air flow inside the box with the lid open. This step is carried out by placing the chamber in a ducted BYPASS fume hood—Phoenix Controls Corporation—100 lfm. All experiments are conducted in this fume hood for the sake of safety. It is noted that the box is sealed during the experiments to prevent any interference of the fume hood.

Post-processing of Coupons and APS Box: For the post experiments, the coupons are thoroughly mixed in 15 ml PBS solution using a Fisher Scientific Mini Vortexer lab mixer and 100 μl of this mixture (for exposed coupons) or its dilution (for unexposed coupons) is plated on agar plates (LB agar for E. coli and Nutrient Agar for B. subtilis) followed by incubation at 37° C. (E. coli) or 30° C. (B. subtilis) for 24 hours. Plate counts are obtained to quantify the bacterial colonies present in the coupons. All post processing is conducted in a Biological Safety Cabinet (BSC) Class II, Type A2 to inhibit external contamination and maintain safety protocols.

Control Experiments: Control experiments are performed to ensure correct quantification of inactivation of bacteria due to the APS and rule out inactivation due to environmental factors related to the experimental setup or procedure. This step involves establishing consistent bacterial concentrations for the sterilized coupons inoculated with a fixed volume of the bacterial culture. For the experiments, inoculated coupons are left inside the APS for periods matching exposure times without powering up the CPPRS and post processed. At least three repeats are performed.

Exposure Experiments: Each exposure experiment involves 14 coupons of one material inoculated with 10 μl of bacteria culture containing approximately 10⁷ CFUs/ml of one type of bacteria. Inoculation volume of 10 μl is selected to obtain a concentration of approximately 10⁵ CFUs/coupon. Then, 11 coupons are placed in the APS at the 11 measurement points shown in FIG. 4 and exposed for selected times. The remaining 3 coupons are placed outside the chamber for the same times as controls. After the exposure periods, all 14 coupons are post-processed to obtain the concentration in terms of CFUs/coupon. The following equation is used to calculate CFUs/coupon:

CFUs/coupon=CFUs/ml*V1=Dx*10^{x}*V1;
where V1=volume of PBS is used to mix coupons in post processing in mls, and Dx=CFUs counted in x^{th} dilution plate.

The reduction in microbial colonies obtained per coupon at each measurement point is determined from the difference in CFUs/coupon of the exposed and control (unexposed) coupons.

### Ozone Measurements

The 2B Technologies Model 106-6 Ozone Monitor, which is operated based on UV light absorption in a range between 180 nm and 280 nm, preferably at 254 nm, is used for the ozone measurements in the APS prototype chamber. The accuracy of the monitor is 0.01 ppm or 2% of the reading. Ozone measurements are performed at the center of the sterilization box for the exposure times determined in the sterilization efficacy tests with corresponding CPPR ON and OFF times.

### Ozone Penetration Tests

An enclosure is built to assess CPPR generated ozone permeability through fabric layers with and without the assistance of an external agent such as a pump. As shown in FIG. 5, the enclosure has the following components: 1) CPPR support tabs to hold the CPPR; 2, 3) top and bottom measurement holes positioned above and below the fabric layer, respectively, for accessing the data collecting probe; 4) air exit hole connected to the pump or left open based on the experiment, and 5) fabric sample holder to hold the fabric samples or cells. A CPPR with the fan reactor panel is utilized for the tests. The fabric sample holder has a rectangular cutout with Velcro attached to Velcro on the boundary of the fabric samples. Ozone concentrations are measured above and below the fabric layers for 5 minutes after 10 seconds of powering up the CPPRs to determine the ozone permeability. Measurements are averaged over the last minute of the measurement period when ozone concentrations are stabilized. Top and bottom measurements are performed separately to inhibit the effects of measurement on data collection. The top and bottom of the enclosure are blocked to mitigate generated ozone from escaping. Before every experiment, it is ensured that ozone levels inside the enclosure is equal to ambient ozone levels. Three repeats are performed for each fabric sample type with and without the pump for forced penetration.

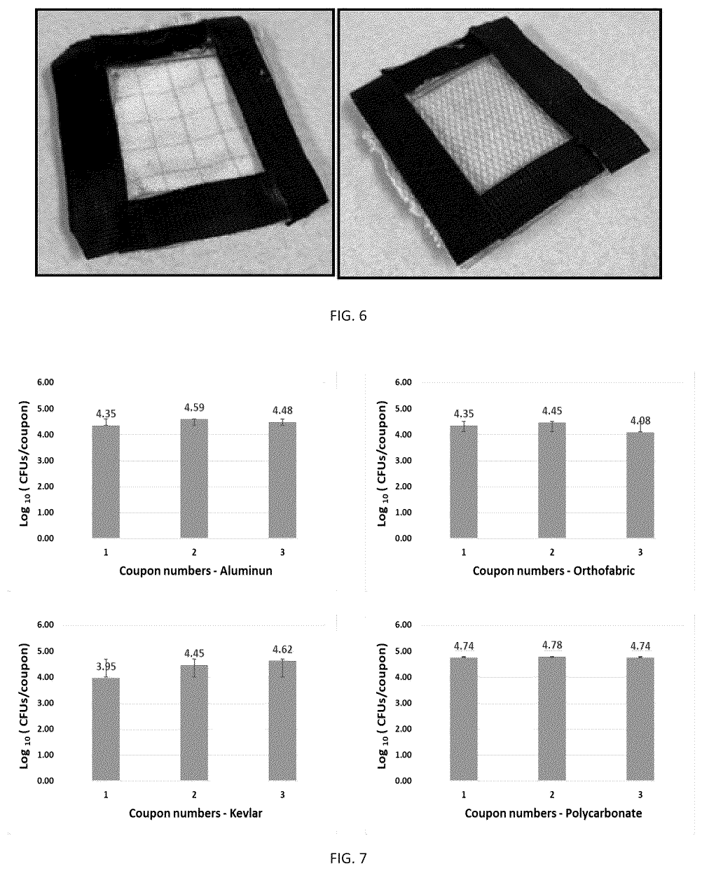

Three fabric types are tested: Kevlar, Orthofabric, and a combined layer of Kevlar and Orthofabric. Velocities and ozone data are collected before and after the fabric layers, with and without using a pump. The samples measure 4 cm×5 cm in dimensions and have thicknesses of 1 mm, 2 mm and 3 mm for Orthofabric, Kevlar and combined samples, respectively. The Kevlar fabric has a thickness of 2 mm and the Orthofabric has a thickness of 1 mm. The samples are held in “cells” made of Velcro which hold the fabric and keep it in a uniform shape as shown in FIG. 6.

Ozone is measured using the 106-6 Ozone Monitor by 2B Technologies as mentioned before. Velocities and temperature are measured using a Testo 405i Smart Probe Hotwire anemometer with an accuracy of +/−0.1 m/s+5% for the measured velocities and +/−0.5° C. for the measured temperature.

Before performing the tests, temperature, humidity, and velocities of air through the chamber both with and without the pump are measured without powering the CPPR. Upstream and downstream air velocities refer to the velocities of the air above and below the fabric layer, respectively. For each reading, a total of 5 measurements are taken and then averaged to get the following values: Temperature=24.4° C., Humidity=62.70%, Upstream Air Velocity with pump=Vₚᵤ=0.08 m/s+/−0.02 m/s, Downstream Air Velocity with pump=V_{pd}=0.15 m/s+/−0.03 m/s, Upstream Air Velocity no pump=Vₙᵤ=0.03 m/s+/−0.01 m/s, and downstream Air Velocity no pump=Vₙᵤ=0.02 m/s+/−0.01 m/s.

### Preliminary Material Compatibility Tests

The four selected materials include aluminum, polycarbonate, Kevlar and Orthofabric, are exposed in the APS for exposure conditions, resulting in complete killing of E. coli and B. subtilis. These exposure conditions correspond to equivalent ozone CT (concentration×time) values required for sterilization (4 to 5 log reduction) of the two test species. All four materials are tested for visible surface degradation and change in material composition using standard SEM analysis. The SEM Hitachi S 3000, at the Nanoscale Research Facility (NRF) at the University of Florida is used to perform these tests.

### Results and Discussion

### Evaluation of Efficacy of the APS in Sterilizing Contaminated Materials of Interest:

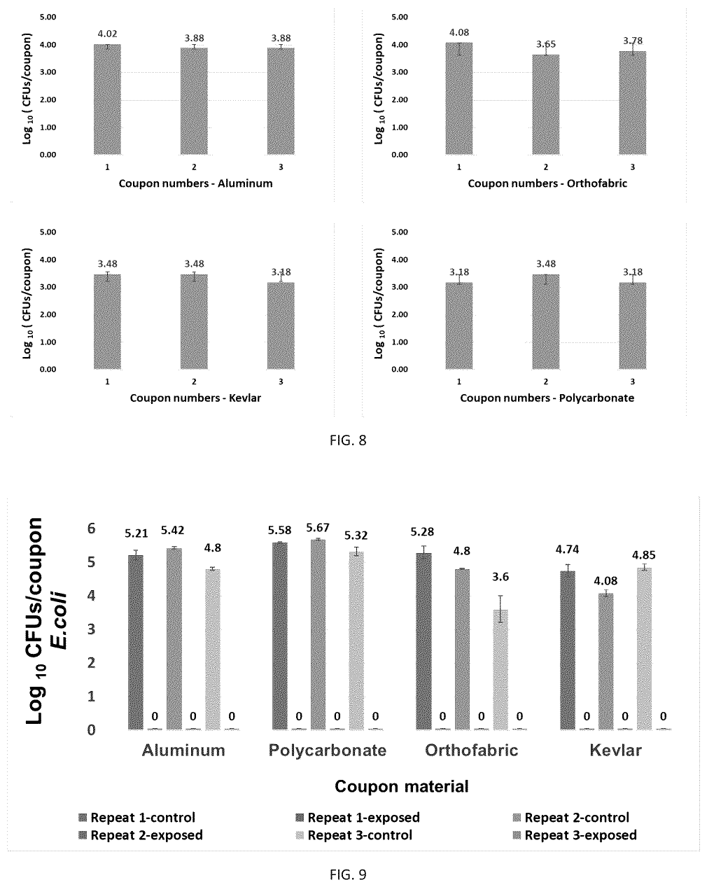

Control experiments: The results obtained for establishing consistent bacterial concentrations for the sterilized coupons of different material inoculated with 5 to 6 logs of E. coli and B. subtilis CFUs are shown in FIGS. 7 and 8 with error bars based on standard deviation from 3 repeats. The data show that inoculating coupons of a same size and a same material with a same amount of culture result in consistent bacterial count per coupon with a maximum variation of 0.3 log₁₀ (CFU/coupon). The data also validate the post processing method to recover microbial population from inoculated coupons.

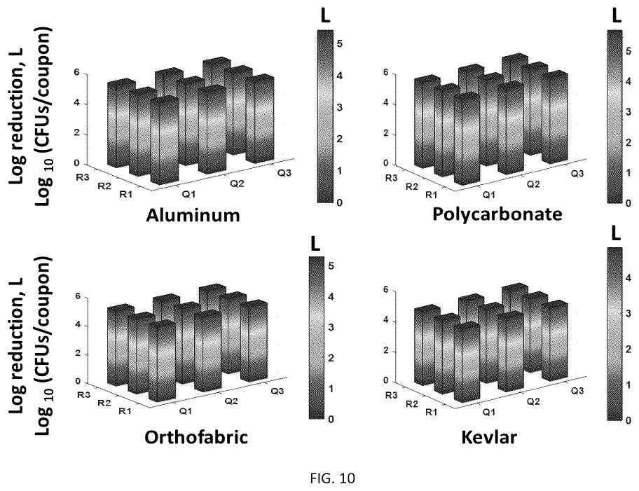

Exposure Experiments: A total of 37 iterative exposure experiments are performed with two test organisms, 7 exposure time points, and 4 selected materials as mentioned in the Materials and Methods section. The iterative experiments are designed to find the optimum combination of the number of CPPRs and exposure time required for complete killing of each test organisms for all selected material within 30 minutes. The results show that with a control count of 4 to 5 logs of CFU/coupon, complete killing for E. coli is achieved with 4 CPPRs in 20 minutes. The same results are achieved for B. subtilis with 6 CPPRs in 30 minutes. FIGS. 9 and 10 show the results of the overall sterilization (average reduction over 11 points of measurement) and spatial distribution of log reductions achieved for each material type contaminated with E. coli and B. subtilis and subjected to APS exposures corresponding to (a) 20 minutes exposure time with 4 CPPRs (15 mins ON+5 mins OFF) and (b) 30 minutes exposure time with 6 CPPRs (25 mins ON+5 mins OFF).

Referring to FIGS. 9 and 10, the results obtained for E. coli contaminated surfaces show that the APS achieves complete killing or 4 to 5 log reductions of E. coli on aluminum, polycarbonate, Orthofabric and Kevlar, at 11 points inside the chamber, with a minimum exposure time of 20 minutes with 4 CPPRs active for 15 minutes.

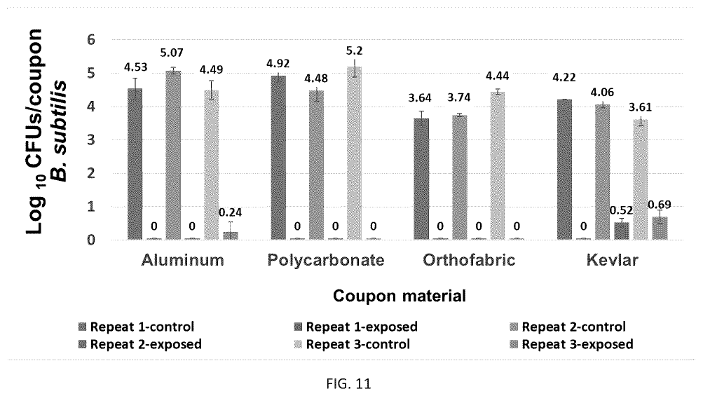

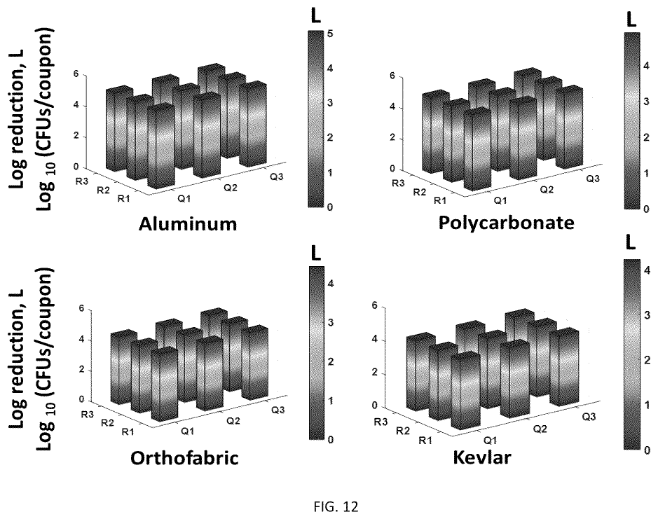

Similarly, FIGS. 11 and 12 show that the APS achieves complete killing of B. subtilis on aluminum, polycarbonate, Orthofabric and Kevlar, at 11 points inside the chamber, with a minimum exposure time of 30 minutes with 6 CPPRs active for 25 minutes. Compared to E. Coli, additional CPPRs and exposure times required for complete killing ofB. subtilis can be explained by B. subtilis being a Gram, positive bacteria with protective cell wall outside the cell membrane, as it has a cytoplasmic membrane and a thick cell wall. Further, as a spore-forming bacteria, the selected B. subtilis Strain can form a tough, protective endospore for resistance, allowing it to tolerate extreme environmental conditions.

The sterilization efficacy results show the potential of APS in uniformly sterilizing an object made of aluminum, Orthofabric, polycarbonate or Kevlar, contaminated with 4 to 5 logs of pathogens like E. coli and B. subtilis, per square inch of surface, within 30 minutes. Further, successful sterilization achieved for the four materials establishes that the APS is capable of sterilizing both solid and porous surfaces without the need of an external mixing or distributing agent.

### Ozone Data and CT (Concentration×Time) Requirements:

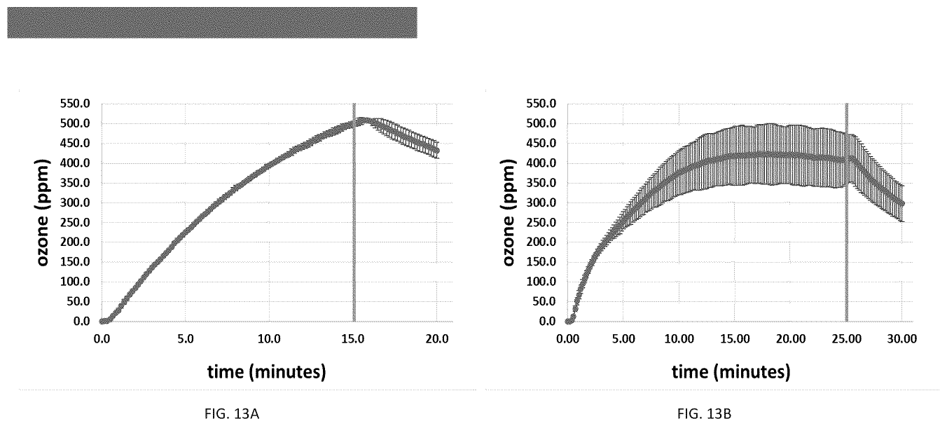

Based on the number of CPPRs and corresponding exposure times that result in complete killing of 4 to 5 logs of E. coli and B. subtilis concentrations on 11 coupons inside the APS chamber, ozone data are collected in the APS at the center point of the central plane (P2) for the following operating conditions: (a) 4 CPPRs and 20 minutes (15 min CPPRs on, 5 mins CPPRs off) and (b) 6 CPPRs and 30 minutes (25 min CPPRs on, 5 mins CPPRs off). These measurements are performed separately from the decontamination experiments to (a) inhibit incorrect measurements due to loss of concentrations measured because of usage of ozone for decontamination, and (b) inhibit contamination of the ozone monitor. The results are shown in FIGS. 13A and 13B with the orange line representing the time when the CPPRs are turned off. Three repeats are performed for each exposure time to gain statistical confidence and standard deviation observed in those repeats are used to represent error bars in the graph.

FIG. 13A shows that the ozone concentrations reach a peak of around 500 ppm in 15 minutes at the center of the APS and gradually decrease thereafter when 4 CPPRs are powered for 20 minutes (CPPRs on for 15 minutes and CPPRs off for 5 minutes), while FIG. 13B shows that when 6 CPPRs are powered for 30 minutes (CPPRs on for 25 minutes and CPPRs off for 5 minutes), the ozone concentrations at the center of the APS converge to around 420 ppm in 15 minutes and stabilize at that concentration till the CPPRs are turned off. Although this seems counter-intuitive, possible explanations for this difference is that (a) with 6 CPPRs distributing the generated ozone, more mixing of ozone occurs inside the APS which can help to better decontaminate a larger volume but can also affect ozone measurements or result in faster decomposition of ozone, and (b) the peak concentration at the center of the APS might shift to another point with more mixing and vortical structures formed due to interaction of flow structures generated by the 6 CPPRs. Furthermore, increased deviations observed in the concentrations with 6 CPPRs powered up in the APS can be attributed to increased mixing and unstable flow structures.

It is noted that the assessment of spatial ozone distribution within the APS is not within the scope of this study, mainly due to time constraints and primary emphasis of this study is placed on investigating the spatial distribution of decontamination within the APS.

Ozone CT requirements are calculated using following formula to account for difference in CPPR on and off times during an exposure:

CT value=Sum(Cᵢtₛ)
where Cᵢ refers to i^{th} sample reading given by the ozone monitor, tₛ refers to sampling time of the ozone monitor, and n is the total number of samples collected during a specific exposure time. The calculated CT values along with maximum ozone concentrations achieved for each exposure time is given below in Table 2.

TABLE 2
Ozone CT (concentration × time) Values Recorded in the
APS for Number of CPPRs and Exposure times of interest.

| | | Ozone CT values |
|---|---|---|
| Exposure time (minutes) | No. of CPPRs | (ppm-min) |
| 20 (15 min CPPR on + | 4 | 6789.02 |
| 5 min CPPR off) | | |
| 30 (25 min CPPR on + | 6 | 10323 |
| 5 min CPPR off) | | |

### Power Consumption:

Based on the sterilization efficacy tests, 6 CPPRs used in a particular embodiment of the APS can achieve complete killing of pathogenic bacteria on various surfaces within 30 minutes with 6 CPPRs active for 25 minutes. With the power requirement of a single CPPR being 2.2±0.37 Watts, the power requirement of the CPPRs in the APS amounts to 13.2±2.22 W.

### Ozone Penetration:

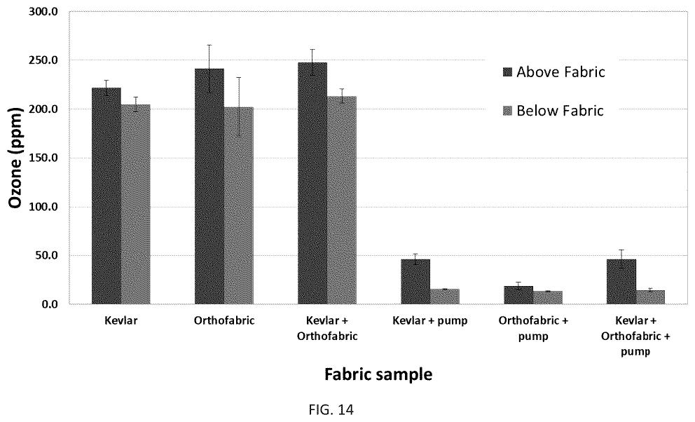

Averaged ozone data over 5 repeats of measurements above (upstream) and below (downstream) fabric layers with and without using a pump, is given in Table 3 below and FIG. 14. Without using the pump, the percentage reduction in ozone concentration from above the fabric layer (the side where the CPPR is placed) to below the fabric sample is 7.59% for Kevlar, 16.17% for Orthofabric and 13.90% for a combined layer of Kevlar and Orthofabric. The results indicate that the CPPR generated ozone can penetrate through the fabric layers in an enclosure without the aid of an external agent. Data collected with the pump show much lower ozone concentrations. This can be attributed to rapid displacement of ozone generated due to suction of the air pump used. The percentage in ozone reduction when using the pump is 66.52% for Kevlar, 28.72% for Orthofabric, and 68.30% when using both fabrics combined. Higher percentage reductions observed when using the pump can be explained by the very low ozone concentrations measured before penetration. Thus, the data shows that a pressure driven mechanism is not necessary for CPPR generated ozone penetration through fabric layers. The data combined with decontamination data for Kevlar and Orthofabric indicate that the APS does not require any additional component for ozone penetration through fabrics such as Kevlar and Orthofabric.

TABLE 3
Data for Ozone Penetration Through Different
Fabric Layers with and without a Pump

| | No Pump | |
|---|---|---|
| | Upstream Velocity: | Downstream Velocity: |
| | 0.03 m/s | 0.02 m/s |
| | Temperature ° C. | |
| | Upstream | Downstream |
| | Temperature Range: | Temperature Range: |
| | 25.3-26.8 | 25.2-26.0 |
| | Average Ozone B | Average Ozone A |
| Fabric | (ppm) | (ppm) |
| Kevlar | 221.7 | 204.9 |
| Orthofabric | 241.5 | 202.4 |
| Kevlar + Orthofabric | 247.7 | 213.3 |
| | Pump Used | |
| | Upstream Velocity: | Downstream Velocity: |
| | 0.08 m/s | 0.15 m/s |
| | Temperature ° C. | |
| | Upstream | Downstream |
| | Temperature Range: | Temperature Range: |
| | 25.1-26.3 | 24.9-25.9 |
| | Average Ozone B | Average Ozone A |
| Fabric | (ppm) | (ppm) |
| Kevlar | 46.3 | 15.5 |
| Orthofabric | 18.8 | 13.4 |
| Kevlar + Orthofabric | 46.3 | 14.7 |

### Material Compatibility:

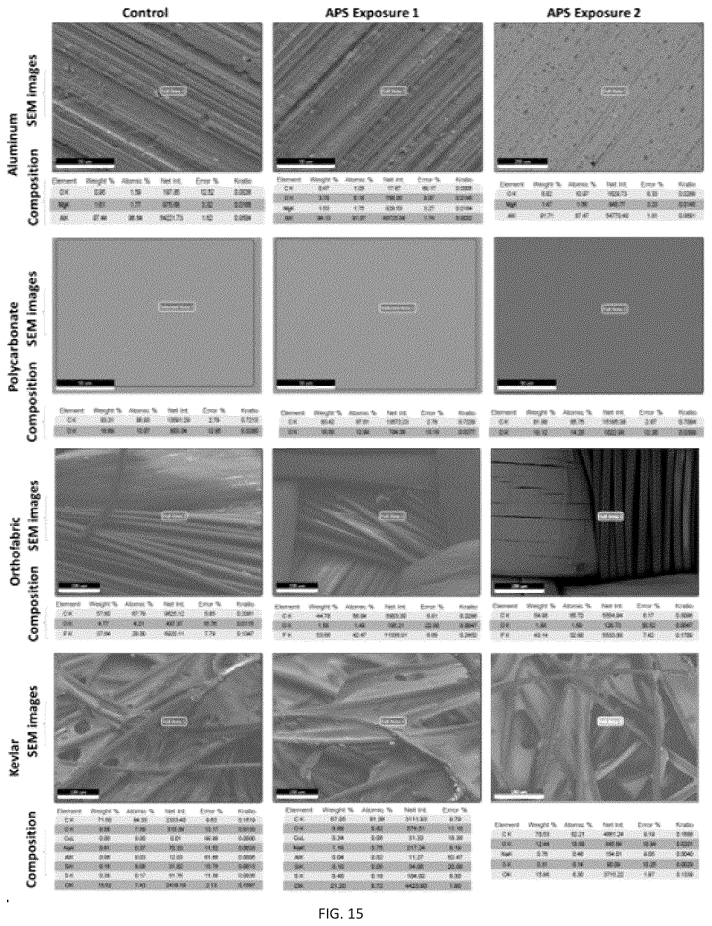

One coupon of all four selected materials: aluminum, polycarbonate, Kevlar and Orthofabric, is exposed in the APS sterilization chamber for the two exposure conditions, resulting in complete killing of E. coli and B. subtilis, respectively: (a) APS Exposure 1: 20 minutes with 4 CPPRs (15 min on+5 min off) and (b) APS Exposure 2: 30 minutes with 6 active CPPRs (15 min on+5 min off). All four materials are tested for visible surface degradation and change in material composition by comparing with a control coupon not exposed to the APS, based on standard SEM analysis. The results are shown in FIG. 15. Data for control (unexposed) material samples are compared with the samples undergoing APS Exposure 1 and APS exposure 2. Visual analysis of the SEM images, at 50 to 200 microns scale, does not show any material degradation. Comparison of material composition shows increase in oxygen content by around 3% for aluminum, 0% for polycarbonate, and 1% for Kevlar for APS Exposure 1. Corresponding increase for APS Exposure 2 is observed to be about 6% for aluminum, about 2% for polycarbonate, and about 5% for Kevlar. These preliminary results suggest that APS exposures required for sterilization does not lead to considerable material degradation for aluminum, polycarbonate, Orthofabric, and Kevlar material. Further SEM analysis with detailed compositional study can be performed for the APS.

### Effectiveness of Active Plasma Sterilizer (APS):

The compact and energy efficient sterilization system of the subject invention having inbuilt ozone generation and mixing capability is provided for sterilization pertaining to planetary protection. Design of sterilization box of the APS prototype and integration of CPPRs into it for ozone generation and distribution is discussed. The CPPRs are integrated based on strategic selection and placement of SDBD reactor panel designs (Fan and Comb reactor configurations) to obtain uniform distribution of the generated ozone through the sterilization box, achieving spatially distributed decontamination based on a synergistic combination of SDBD ozone generation and flow actuation for distributing and mixing DBD generated ozone without using external mixing agents or moving parts. The APS prototype is evaluated by (a) testing its sterilization efficacy for coupons made of 4 material types (aluminum, polycarbonate, Kevlar, Orthofabric), contaminated with 4 to 5 logs of two test organisms: (E. coli and B. subtilis), and distributed at 11 points inside the APS, (b) determining the number of CPPRs required for complete killing of selected organisms within 30 minutes, (c) determining corresponding ozone CT (concentration×time) requirements and power requirements, (d) testing ozone penetration through selected fabric materials, and (e) performing preliminary material compatibility tests through SEM analysis. Results show that the APS can achieve 4 to 5 log reductions of pathogenic bacteria such as E. coli and B. subtilis on materials including but not limited to, aluminum, polycarbonate, Kevlar and Orthofabric, simultaneously at 11 points inside the chamber, with a maximum ozone CT requirement of 10323 ppm-minute, within 30 minutes with total power consumption of 13.2 W. Spatial distribution of the sterilization data at the central plane of the APS reveals that the APS can uniformly sterilize several points on a contaminated surface. Successful CPPR generated ozone penetration for single and combined fabric layers is established with a maximum reduction of 16.17% in ozone concentrations through the layers without using an external agent to enhance penetration. Preliminary material compatibility tests with SEM analysis of 4 selected materials exposed to ozone CT values required for sterilization in the APS show no significant material damage, demonstrating the potential of the APS as a sterilization technology for applications in planetary protection with advantages of uniform spatial decontamination, low processing temperatures, low exposure times, lightweight with no moving parts, ability to decontaminate porous surfaces and compatibility with relevant materials.

According to the embodiments of the subject invention, a safe, compact and energy efficient sterilization system, the Active Plasma Sterilizer, with inbuilt ozone mixing and residual ozone removal systems is provided for sterilization in spacecraft facilities pertaining to planetary protection. Accordingly, a compact, lightweight, nonthermal, superior safety, cost competitive and rapid decontamination system and methods is developed to be integrated into spacecraft and platform subsystems. Potential applications include sterilization of spacecraft components and ground-based contamination control that can withstand testing operations and inflight component cross-contamination control. Some application examples are sterilization of sample acquisition equipment (for example, drills), surfaces and space suits pre-launch and post-launch. The embodiments of the subject invention extend sterilization modalities beyond the confines of time and temperature, encompassing the cleaning of adhesive surfaces and refining cleaning protocols beyond the utilization of alcohol and bleach, as stated in the recent 2020 NASA Technology Taxonomy report

The following technical accomplishments are achieved: (i) a first Active Plasma Sterilizer (APS) system and methods with integrated Compact Portable Plasma Reactors (CPPRs) for testing are developed; (ii) sterilization (4 to 5 log reduction) of aluminum, polycarbonate, Kevlar and Orthofabric coupons contaminated with E. coli and B. subtilis, simultaneously at 11 points distributed inside the prototype, within 20 to 30 minutes; (iii) the number of CPPRs in the APS required for sterilization within 30 minutes is determined to be 6; (iv) the ozone CT requirements for sterilization of E. coli and B. subtilis are determined to be 6789.02 ppm-minute and 10323 ppm-minute, respectively; (v) the power requirements of the APS prototype with 6 CPPRs is found to be 13.2 W.

In one embodiment, the prototype of the APS is determined to be 30 L in volume, even though the size of the APS is scalable.

A total of 37 iterative exposure experiments are performed with two test organisms, 7 exposure time points and 4 selected materials for determining the optimum combination of number of CPPRs and minimum exposure time required for complete killing of each test organisms for all selected material.

The experiment results show that with a control count of 4 to 5 logs of CFU/coupon, complete killing for E. coli is achieved with 4 CPPRs within 20 minutes. The same results are obtained for B. subtilis with 6 CPPRs within 30 minutes.

The results of the overall sterilization and spatial distribution of log reductions achieved for each material type contaminated with E. coli and B. subtilis and subjected to APS exposures corresponding to (a) 20 minutes exposure time with 4 CPPRs active for 15 minutes and (b) 30 minutes exposure time with 6 CPPRs active for 25 minutes, and relevant ozone data are shown. In addition, the report includes pertinent ozone data related to these experiments.

The APS system and methods hold significant commercialization potential, with applications ranging from sanitizing rooms in hospitals and businesses to versatile adaptability in produce preservation and decontamination. Additionally, it offers the capability to decontaminate everyday items, including children's toys, clothing, bags, shoes, and more. Once the product receives technical validation, market drivers will encompass the demand for safe and efficient decontamination of larger equipment and volumes.

According to embodiments of the subject invention, the APS system and methods are lightweight, low-cost, non-thermal and designed for rapid decontamination with potential integration into spacecraft or platform subsystems. It addressed the disadvantages related to the existing methods for space missions such as high processing temperatures and material incompatibility. Additionally, the APS may be used to achieve microbial mitigation in crewed missions. In comparison to large and high-power consuming DBD ozone generation systems that already exist, the APS offers advantages including, but not limited to, (a) compact, lightweight and low energy power supply, and (b) SDBD flow actuation aided ozone mixing leading to rapid decontamination with lower ozone requirements and lower residual ozone. Potential applications also include sterilization of spacecraft components and subsystems, ground-based contamination control that can withstand testing operations and in-flight component cross-contamination control. Certain application examples are sterilization of sample acquisition equipment (for example, drills), surfaces, and space suits pre-launch and post-launch.

### Ozone Decomposition Module (ODM)

Ozone is a potent antimicrobial agent. Ozone destroys microorganisms by reacting with oxidizable cellular components, particularly those containing double bonds, sulfhydryl groups, and phenolic rings. Hence, ozone targets membrane phospholipids, intracellular enzymes, and genomic material, leading to cellular damage and the death of microorganisms. Ozone offers many advantages as a sterilant/disinfectant gas and is a very efficient sterilant due to its strong oxidizing properties (E=2.076). Therefore, ozone has been widely used for sterilization of containers for aseptic packaging, decontamination of fresh produce, and food preservation in cold storage.

Nevertheless, ozone is an inherently unstable gas that has to be produced on-site, as it cannot be stored, making it impractical for use in many settings. Moreover, ozone breaks down to harmless oxygen after just a couple of hours. Nevertheless, in practical ozone applications, there is need for the capability of breaking it down at a much faster rate once its sterilization activity is finished. OSHA regulations require that ozone levels must not exceed 0.1 ppm during an 8-hour exposure period, a concentration significantly lower than what is typically required for most ozone-based applications. The capability to rapidly decompose ozone into harmless oxygen confers numerous advantages to technologies utilizing ozone, reducing the post-use waiting time significantly.

According to embodiments of the subject invention, the ozone decomposition module (ODM) comprises a heat coil, electronics, and piping to provide a path for ozonated air to travel through. The heating coil heats the ozonated air as it passes through. Heating the air increases the decomposition rate of the ozone in the air. The electronics supply power to the heating coil, allowing it to operate with a reasonably low power. An air pump such as a fan is used to force or pull out the ozonated air from the ozone generation chamber through the ODM and recirculated as many times as needed. The flowrate of the air pump can be in a range between 10-3 L/min to 10⁶ L/min.

Embodiments of the subject innovation have demonstrated that for a 5 L with 100 ppm of ozone, the ODM operating at temperatures of 400° C. and a flow rate of 250 L/min can reduce the ozone concentration inside the chamber to lower than 0.1 ppm within 60 seconds. The electrical circuitry for the heating element in the current ODM embodiment uses an induction coil with a ZVS driver. Results with the same setup demonstrate reduction of ozone in the chamber to approximately 1 ppm after 30 seconds.

Embodiments of the subject invention may include systems such as induced air flow (for example, through the use of fans), adjustment of temperature within the heat coil for varying levels of ozone concentration, measurement of ozone inside flow path to automatically adjust usage levels, or other suitable means.

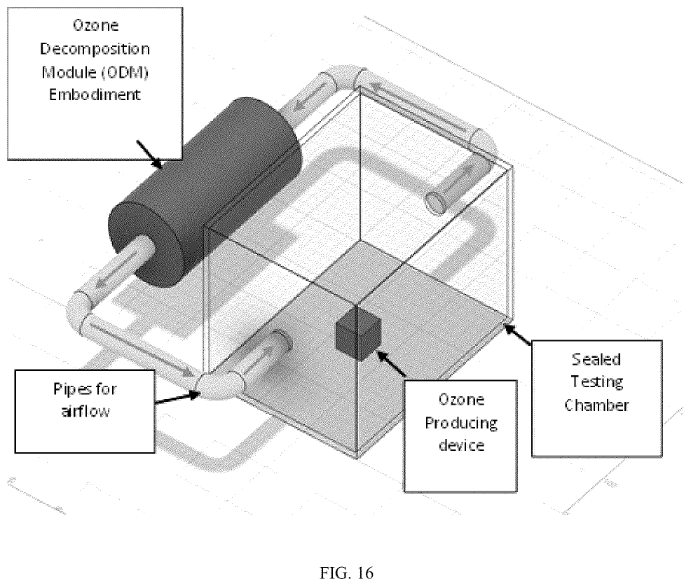

An initial testing setup is developed, as shown in FIG. 16. For the test, an ozone generator is disposed in a chamber to produce 100 ppm of ozone in a space of 5 L volume. The testing procedure is described as follows:
1. Setup the ozone monitor to measure the initial ozone in the chamber.
2. Turn on the CPPR for 6 minutes to reach approximately a concentration of 100 ppm inside the chamber. During the period of time, record the ozone levels in the chamber every seconds using the ozone monitor.
3. Turn the CPPR off and activate the ODM for 0 seconds. During this time, the ozone monitor must be removed or switch off as changes in pressure can cause problems with the ozone monitor.
4. Turn the ODM off after the allotted time and turn the ozone monitor back on. Record the ozone levels inside the chamber for 10 minutes.
5. Open the chamber to allow ozone levels to reach equilibrium with the room levels.
6. Seal the chamber once ozone levels have reached a concentration lower than 0.1 ppm.
7. Repeat steps 1-6 for ODM with activation times of 30 seconds and 60 seconds.

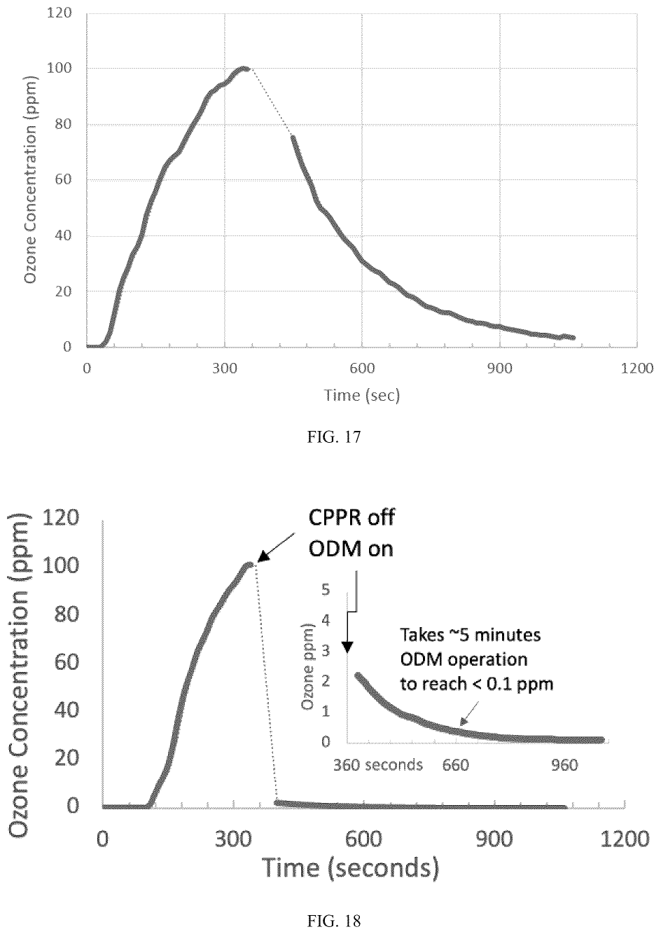

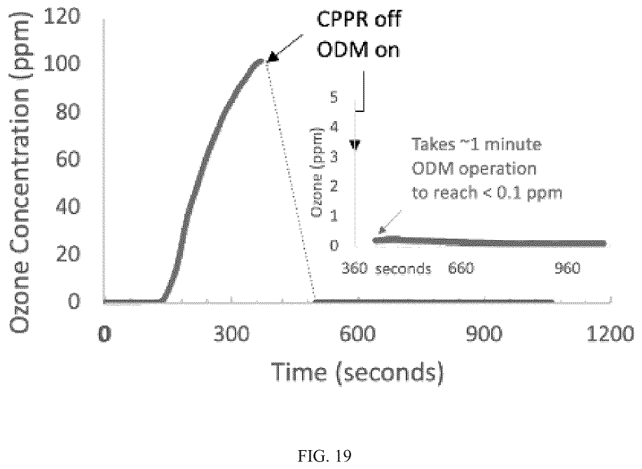

The results from the tests are shown in FIGS. 17, 18, and 19. The 0 second tests show the natural decay rate of ozone inside the chamber as shown in FIG. 17. After 10 minutes, the ozone concentration within the chamber stabilizes at around 3.4 ppm, underscoring the essential role of the Ozone Destruction Module (ODM). Without the ODM, relying on the natural decay of ozone would significantly extend the operational duration of the APS. For 30 and 60 seconds of ODM operation, results are plotted in FIGS. 18 and 19, respectively, showing that as the ODM operation time increases, the ozone inside the chamber decreases by an order of magnitude.

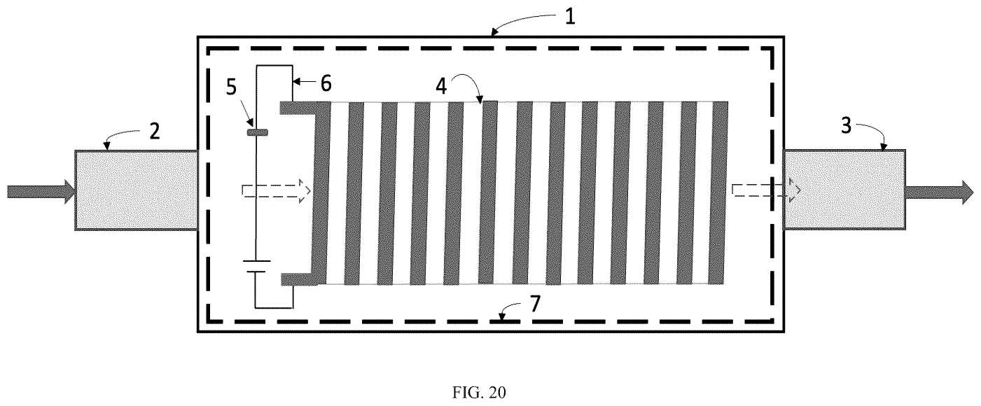

Referring to FIG. 20, in one embodiment, the ODM comprises a casing 1 with inlet and outlet ports 2 and 3, heating element 4, ozone sensor 5, powering circuit 6, and insulation 7. The heating element may be placed internally as a coil, a wire mesh, or other suitable form that can be connected to an electrical source for joule-type heating or may be used as a collector of an external heat source like radiation or e-beam.

In certain embodiment, the ODM comprises a heating element connected to a power supply and configured to decompose ozone by heating ozonated air; a power supply module for supplying power to the heating element; connecting pipes for providing paths for ozonated air to travel through; and an air pump configured to circulate the ozonated air generated. The ODM may additionally comprise one or more sensors configured to measure ozone level, or an insulation element for insulating heat generated by the heating element. The power supply module is a standard wall supply module or a battery powered module. The power supply module additionally comprises feedback loop to supply power to the heating coil. The power supply module may further comprise a power amplifier and a controller configured to control an input voltage to the power amplifier. An input voltage of the power supply module is supplied in a duty cycle. The power supply module may further comprise feedback circuitry configured to monitor an operational parameter of a load and provide feedback to the controller, wherein the controller is configured to control operation of the power amplifier based on the provided feedback. The feedback circuitry comprises an ozone sensor configured to detect ozone in airflow.

In one embodiment, the ozone level is measured (e.g., the at least one sensor is configured to measure the ozone level) based on UV light absorption in a range of from 180 nm and 280 nm, preferably at 254 nm, in the Ozone Destruction Module (ODM).

In one embodiment, the heating element is an induction heating mesh/coil.

In one embodiment, water or other liquid solvent is employed to facilitate the pumping and removing of contaminated air, thereby enhancing the rapid absorption of Reactive Oxygen and Nitrogen Species (RONS).

| Ozone Data | | | ODM TEST |
|---|---|---|---|
| Time (sec) | (in ppm) | | DATA |
| 0 | 0.07 | 0.28 | 0.05 |
| 10 | 0.08 | 0.27 | 0.05 |
| 20 | 0.08 | 0.27 | 0.05 |
| 30 | 0.1 | 0.25 | 0.05 |
| 40 | 1.77 | 0.25 | 0.05 |
| 50 | 5.15 | 0.25 | 0.06 |
| 60 | 11.95 | 0.28 | 0.06 |
| 70 | 20.39 | 0.27 | 0.06 |
| 80 | 24.95 | 0.27 | 0.06 |
| 90 | 28.59 | 0.25 | 0.05 |
| 100 | 33.22 | 0.3 | 0.05 |
| 110 | 36.16 | 1.85 | 0.05 |
| 120 | 40.31 | 5.9 | 0.06 |
| 130 | 47.25 | 9.7 | 0.28 |
| 140 | 52.7 | 13.15 | 1.87 |
| 150 | 55.99 | 17.45 | 5.41 |
| 160 | 60.38 | 24.48 | 9.75 |
| 170 | 64.66 | 33.66 | 15.21 |
| 180 | 66.87 | 42.48 | 23.43 |
| 190 | 68.3 | 49.73 | 32.54 |
| 200 | 69.98 | 55.21 | 39.58 |
| 210 | 73.1 | 60.56 | 44.81 |
| 220 | 76.24 | 65.83 | 50.21 |
| 230 | 79.59 | 69.71 | 56.11 |
| 240 | 82.18 | 73.71 | 61.39 |
| 250 | 84.73 | 78.38 | 66 |
| 260 | 89.04 | 81.67 | 70.29 |
| 270 | 91.36 | 84.62 | 74.58 |
| 280 | 92.36 | 87.75 | 78.94 |
| 290 | 93.85 | 90.46 | 82.39 |
| 300 | 94.56 | 92.53 | 85.22 |
| 310 | 95.61 | 94.98 | 88.56 |
| 320 | 97.73 | 98.13 | 91.58 |
| 330 | 99.4 | 100.7 | 93.83 |
| 340 | 99.9 | 101.2 | 96.44 |
| 350 | 99.77 | 102.1 | 99.29 |
| 360 | ODM OFF | ODM Turned ON | |
| 370 | | | |
| 380 | | | |
| 390 | | 2.25 | |
| 400 | | 2.21 | |
| 410 | | 2.07 | |
| 420 | 85.19 | 1.95 | 0.22 |
| 430 | 81.14 | 1.79 | 0.22 |
| 440 | 77.25 | 1.67 | 0.21 |
| 450 | 75.19 | 1.55 | 0.22 |
| 460 | 69.46 | 1.43 | 0.21 |
| 470 | 64.91 | 1.32 | 0.23 |
| 480 | 61.74 | 1.23 | 0.22 |
| 490 | 57.73 | 1.15 | 0.21 |
| 500 | 52.69 | 1.07 | 0.2 |
| 510 | 49.84 | 1 | 0.21 |
| 520 | 48.25 | 0.92 | 0.21 |
| 530 | 46.57 | 0.88 | 0.22 |
| 540 | 44.06 | 0.84 | 0.23 |
| 550 | 41.11 | 0.81 | 0.23 |
| 560 | 38.98 | 0.77 | 0.23 |
| 570 | 37.41 | 0.71 | 0.21 |
| 580 | 35.55 | 0.65 | 0.21 |
| 590 | 32.97 | 0.6 | 0.2 |
| 600 | 30.95 | 0.57 | 0.2 |
| 610 | 29.65 | 0.53 | 0.19 |
| 620 | 28.1 | 0.49 | 0.19 |
| 630 | 27.12 | 0.47 | 0.19 |
| 640 | 26.65 | 0.44 | 0.18 |
| 650 | 24.86 | 0.41 | 0.18 |
| 660 | 23.07 | 0.38 | 0.17 |
| 670 | 22.53 | 0.36 | 0.16 |
| 680 | 21.5 | 0.34 | 0.16 |
| 690 | 19.8 | 0.32 | 0.16 |
| 700 | 18.56 | 0.29 | 0.14 |
| 710 | 17.85 | 0.27 | 0.14 |
| 720 | 17.12 | 0.26 | 0.14 |
| 730 | 15.85 | 0.24 | 0.14 |
| 740 | 14.69 | 0.23 | 0.13 |
| 750 | 14.15 | 0.21 | 0.13 |
| 760 | 13.47 | 0.2 | 0.12 |
| 770 | 12.74 | 0.18 | 0.12 |
| 780 | 12.5 | 0.18 | 0.12 |
| 790 | 12.26 | 0.16 | 0.12 |
| 800 | 11.74 | 0.15 | 0.12 |
| 810 | 10.94 | 0.15 | 0.11 |
| 820 | 10.16 | 0.14 | 0.11 |
| 830 | 9.69 | 0.13 | 0.11 |
| 840 | 9.28 | 0.13 | 0.1 |
| 850 | 8.78 | 0.12 | 0.1 |
| 860 | 8.5 | 0.11 | 0.09 |
| 870 | 8.31 | 0.11 | 0.09 |
| 880 | 7.83 | 0.11 | 0.09 |
| 890 | 7.52 | 0.11 | 0.09 |
| 900 | 7.28 | 0.11 | 0.08 |
| 910 | 6.86 | 0.11 | 0.08 |
| 920 | 6.57 | 0.11 | 0.09 |
| 930 | 6.3 | 0.11 | 0.09 |
| 940 | 6.01 | 0.1 | 0.1 |
| 950 | 5.64 | 0.09 | 0.09 |
| 960 | 5.18 | 0.09 | 0.09 |
| 970 | 4.78 | 0.09 | 0.09 |
| 980 | 4.59 | 0.09 | 0.09 |
| 990 | 4.4 | 0.09 | 0.09 |
| 1000 | 4.19 | 0.09 | 0.08 |
| 1010 | 4.02 | 0.09 | 0.08 |
| 1020 | 3.69 | 0.09 | 0.09 |
| 1030 | 3.41 | 0.09 | 0.09 |
| 1040 | 4.02 | 0.09 | 0.09 |
| 1050 | 3.69 | 0.09 | 0.09 |
| 1060 | 3.41 | 0.09 | 0.09 |

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. An active plasma sterilizer (APS) system for sterilization/decontamination, comprising:
a sterilization box; and
a plurality of compact portable plasma reactors (CPPRs) disposed in the sterilization box and configured for generating surface dielectric barrier discharge (SDBD) for generating and distributing reactive oxygen and nitrogen species (RONS).

2. The APS system according to claim 1, wherein the sterilization box is made of polycarbonate.

3. The APS system according to claim 1, wherein the sterilization box comprises a grid structure disposed inside the sterilization box and configured for decontamination measurement.

4. The APS system according to claim 1, wherein the sterilization box comprises a plurality of through-holes disposed on lateral sides of the sterilization box, allowing suspension of inoculated coupons inside the sterilization box.

5. The APS system according to claim 1, wherein the RONS includes ozone.

6. The APS system according to claim 1, wherein each of the plurality of CPPRs comprises a reactor panel and a power supply circuit.

7. The APS system according to claim 6, wherein the reactor panel comprises one or more electrodes separated by a dielectric medium.

8. The APS system according to claim 6, wherein the power supply circuit is configured to convert a low DC voltage to a high AC voltage for generating SDBD on surfaces of the reactor panel.

9. The APS system according to claim 6, wherein the plurality of CPPRs comprise two reactors panels configured to generate different flow actuation for better distribution of the generated SDBD in the sterilization box.

10. The APS system according to claim 9, wherein the two reactor panels include a comb reactor panel and a fan reactor panel, wherein the comb reactor panel is configured to generate a two-dimensional (2D) flow distribution with a dominant wall jet in a direction from a shaft towards teeth tips of the comb reactor panel, and wherein the fan reactor panel is configured to generate a three-dimensional (3D) flow distribution forming a swirl flow spreading vertically upwards and outwards from a center of the fan reactor panel.

11. The APS system according to claim 10, wherein the plurality of CPPRs comprise three CPPRS each comprising a fan reactor panel and the three fan reactor panels are positioned in an equilateral triangle formation on a top internal surface of the sterilization box, and one CPPR comprising a comb reactor panel disposed at a bottom surface of the sterilization box with a predetermined distance offset from a center of the bottom surface.

12. The APS system according to claim 1, further comprising at least one sensor and feedback circuitry configured to monitor a voltage or an operational parameter of a load and provide feedback to a controller to control the voltage or inhibit current overload, wherein the controller is configured to control operation of a power amplifier based on the provided feedback.

13. The APS system according to claim 1, further comprising at least one sensor to measure temperature, humidity, and/or ozone amount of air for optimized operation.

14. The APS system according to claim 1, further comprising a door lock for safety of operation of the APS system, wherein the door lock is an electromagnetic door lock, a mechanical door lock, or an electromagnetic and mechanical door lock.

15. The APS system according to claim 1, further comprising a wireless or wire linked device for data logging.

16. An ozone decomposition system, comprising:
a heating element connected to a power supply and configured to decompose ozone by heating ozonated air;
a power supply module for supplying power to the heating element;
connecting pipes for providing paths for ozonated air to travel through; and
an air pump configured to circulate the ozonated air generated.

17. The ozone decomposition system according to claim 16, further comprising at least one sensor configured to measure ozone level.

18. The ozone decomposition system according to claim 17, wherein the at least one sensor is configured to measure the ozone level is measured based on UV light absorption in a range of from between 180 nm and to 280 nm.

19. The ozone decomposition system according to claim 16, further comprising an insulation element for insulating heat generated by the heating element.

20. The ozone decomposition system according to claim 16, wherein the power supply module is a standard wall supply module or a battery powered module.

21. The ozone decomposition system according to claim 16, wherein the power supply module comprises feedback loop to supply power to the heating coil.

22. The ozone decomposition system according to claim 21, wherein the power supply module further comprises a power amplifier and a controller configured to control an input voltage to the power amplifier.

23. The ozone decomposition system according to claim 22, wherein an input voltage of the power supply module is supplied in a duty cycle.

24. The ozone decomposition system according to claim 23, wherein the power supply module further comprises feedback circuitry configured to monitor an operational parameter of a load and provide feedback to the controller, wherein the controller is configured to control operation of the power amplifier based on the provided feedback.

25. The ozone decomposition system according to claim 16, wherein the heating element is an induction heating mesh/coil.

26. An active plasma sterilizer (APS) system for sterilization/decontamination, comprising:
a sterilization box;
a plurality of compact portable plasma reactors (CPPRs) disposed in the sterilization box and configured for generating surface dielectric barrier discharge (SDBD) for generating and distributing reactive oxygen and nitrogen species (RONS); and
the ozone decomposition system according to claim 16.

27. The active plasma sterilizer (APS) system according to claim 26, wherein when the ozonated air is contaminated, the contaminated air is removed by a liquid solvent for enhanced absorption of the reactive oxygen and nitrogen species (RONS).
